(19) United States
(12) Patent Application Publication
(10) Pub. No.: US 2021/0024865 A1
(43) Pub. Date: Jan. 28, 2021

(54) SYSTEM FOR OBSERVATION OF MEDIA DISSOLUTION AND/OR BACTERIAL GROWTH IN A TRANSPARENT BAG

(71) Applicant: Merck Patent GmbH, Darmstadt (DE)

(72) Inventors: Stephane OLIVIER, Rosheim (FR); Gaetan BOUR, Ostwald (FR)

(73) Assignee: Merck Patent GmbH, Darmstadt (DE)

(21) Appl. No.: 17/040,460

(22) PCT Filed: Mar. 20, 2019
(86) PCT No.: PCT/EP2019/056909

§ 371 Date: Sep. 22, 2020

(30) Foreign Application Priority Data
Mar. 23, 2018 (EP) 18290022.5

(51) Int. Cl. C12M 1/00; C12M 1/34

(52) U.S. Cl. CPC C12M 23/14; C12M 21/02; C12M 41/36; C12M 41/10

(57) ABSTRACT

The invention provides a system for observation of media dissolution and/or bacterial growth in a transparent bag, comprising a receptacle for supporting the bag, and a light emitting device arranged to transmit light into and/or through the interior of the bag supported at the receptacle. The media in the transparent bag can be visually inspected to confirm the dissolution state of the media and check for residual undissolved media and/or confirm the absence of unexpected microbial growth without having to move or handle the bag as it can remain supported in a defined orientation at the receptacle during repeated visual inspections by means of the light emitted from the light emitting device

This invention concerns a system for observation and preferably monitoring of media dissolution and/or bacterial growth in a transparent/opaque bag. This invention is applicable in particular in the fields of food and beverage, biopharmaceutical, cosmetics, hospital, but also for diagnostic, healthcare and research for instant media preparation.

In the above fields liquid media are often prepared and stored in bottles, vials or bags. The media can be in the form of a concentrated liquid solution or a powder or a granulated material which is dissolved and/or mixed with a liquid, normally water or a solvent. In case of liquid media stored in a clear rigid container, the turbidity of the media is visually checked before use in order to check the progress of dissolution and/or the sterility of (bacteria growth in) the media. In case of liquid media in a small bag, the turbidity of the media is usually checked by holding the bag vertically against a bright background in order to see through the bag.

Visual inspections of this type require repeated manual handling of the respective containers which normally requires that other work or handling processes are interrupted. The inspections distract a user from other tasks and are time consuming. In particular where the bags are large, the bags must remain laid flat and cannot be easily moved to see through the bag. Lifting the bag is feasible for small bags (3 liter) but could be an issue if large bags of 10 liter or more are used. The visual inspections by users can be inconsistent or even erroneous due to varying light conditions or other human factors.

Repeated observation and monitoring is necessary in order to determine complete dissolution or rehydration of the media in the container in order to be able to confirm that the media is ready to be used. Certain dissolution processes also require a mixing operation to promote dissolution. The visual inspection is to determine the point of time when the mixing operation is to be stopped.

The invention accordingly aims at providing a system that allows the user to know efficiently and reliably that a dissolution process is completed and/or whether bacterial growth occurred. It is accordingly an object of the present invention to provide a system for observation and preferably monitoring of media dissolution and/or bacterial growth in a transparent/opaque bag which is efficient, reliable, safe and can be realized at low cost.

In order to solve that problem the present invention provides a system for observation of media dissolution and/or bacterial growth in a transparent bag as defined in claim 1. Preferred embodiments are defined in the dependent claims.

The invention accordingly provides, in the broadest concept, a system for observation of media dissolution and/or bacterial growth in a transparent bag, comprising a receptacle for supporting the bag, and a light emitting device arranged to transmit light into and/or through the interior of the bag supported at the receptacle.

The system of the invention provides the advantage that the media in the transparent bag can be visually inspected to confirm the dissolution state of the media and check for residual undissolved media and/or confirm the absence of unexpected microbial growth without having to move or handle the bag as it can remain supported in a defined orientation at the receptacle during repeated visual inspections by means of the light emitted from the light emitting device.

Preferably, the light emitting device is arranged to transmit the light from one or more areas of a supporting side of the receptacle intended to be in contact with the bag to preferably form a backlit surface, or towards one or more areas of the supporting side from the opposite side with the bag placed in-between. The light emitting device can accordingly cover a large area of, preferably the full major surface of the bag arranged at the receptacle. Further, the observations and inspections can be done at the place where the undissolved particles settle or are expected to settle considering the orientation of the bag held at the receptacle and a sufficiently large area of the light emitting device allows that the overall bag interior can be observed.

Preferably, the light emitting device is configured to be adjusted with respect to wavelength and/or intensity of emitted light. Preferably, the light emitting device comprises a filter for determining the light spectrum transmitted into the bag. Thereby, the observation can be optimized according to the volume of the bag and/or the type of media observed and the heat input into the bag from the emitted light can be controlled.

Preferably, the light emitting device comprises an area light source or plural light sources distributed over an area, wherein the light sources preferably include an array of LEDs or an OLED light source.

Preferably, the system comprises a device for actively transmitting heat to and/or from the bag supported at the receptacle, i.e. by convection, radiation and/or conduction. The device for transmitting heat may be the light emitting device itself. This function allows to warm or cool and regulate the temperature of the media content in the bag during the media preparation or during storage for certain periods of time. Examples of a heat transmitting device other than the light emitting device itself are electrical resistance heaters or a heat pipe circulating a heating/cooling medium in the vicinity of the receptacle for the bag.

Preferably, the receptacle is associated to a moving device to impart a motion to the bag and/or the media in the bag supported at the receptacle. The moving device can promote dissolution and avoid stagnant zones in the interior of the bag. It may also avoid local temperature differences due to the heat introduced by the light emitting device.

Preferably, the system comprises a light sensor device arranged to receive at least part of the light transmitted into and/or through the interior of the bag supported at the receptacle. Depending on the location of the sensor device it may thus detect light that is diffused, refracted and/or reflected by the content of the bag. For example, the sensor device may be located on the side of the receptacle in contact with the bag and supporting the bag and/or on an opposite side with the bag placed in-between. The sensor device for the light can be a single sensor or can be an array of multiple sensors. The use of sensor(s) allows an automatic and unattended observation and monitoring of the media dissolution and/or the bacterial growth in the transparent bag without activity and interaction by the user.

The system may also contain a temperature sensor arranged to detect the temperature of the bag and/or of the media in the bag supported on the receptacle.

Preferably, the system comprises a control device configured to receive the output of the light sensor device(s), if provided, and/or of the temperature sensor, if provided, and to analyse the data, preferably over time, and to output a warning signal and/or to visualize the data and/or to control operation of the light emitting device and/or of the device for transmitting the heat to the bag. The inclusion of a control device further supports an unattended automatic operation of the system from an initial placement of a bag at the receptacle of the system until complete dissolution. It also allows, in another mode, the monitoring of the media in the bag and the automatic detection of bacterial growth. The detection results can be informed to the user through a warning signal to alert the user. The detection results can be visualized in any desired form on a display included in the system or on external devices connected, through data exchange, with the system.

The control device may be configured to analyse the light and/or temperature distribution over at least part of the supporting area of the receptacle. Especially in combination with plural light and/or temperature sensors installed to inspect the bag at different positions the control device may generate an evaluation result that allows the user to locate zones in the bag where dissolution is still incomplete or where bacterial growth takes place. The control device may also be connected to the light emitting device and/or the heating device and/or the moving device in order to automatically switch these devices on/off to avoid local overheating or an undesired temperature distribution in the bag or to control the dissolution progress.

The receptacle may include a plate or a grid for supporting the bag. The receptacle of the system may be configured and dimensioned to support a bag having a volume of at least 1 liter, preferably at least 10 liter, preferably at least 20 liter. The provision of a grid between the light emitting device and the bag can reduce the area of contact between the bag and the receptacle and can reduce the introduction amount of heat into the interior of the bag.

The invention will now be described by reference to the attached drawing in which:

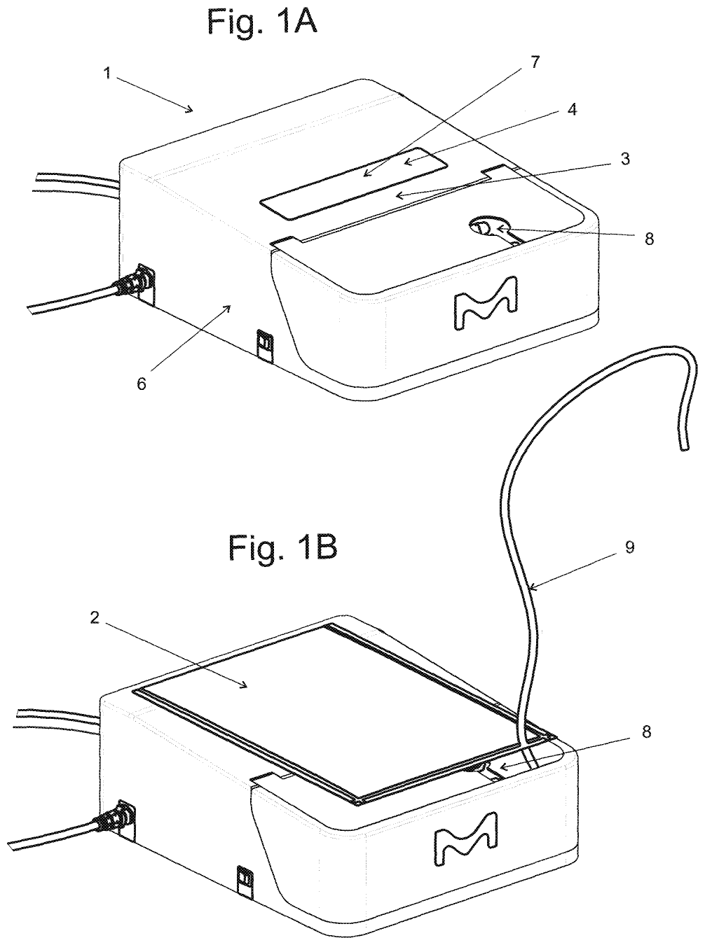

FIG. 1A/B show a first embodiment of a system according to the invention with and without a bag placed on the receptacle; and

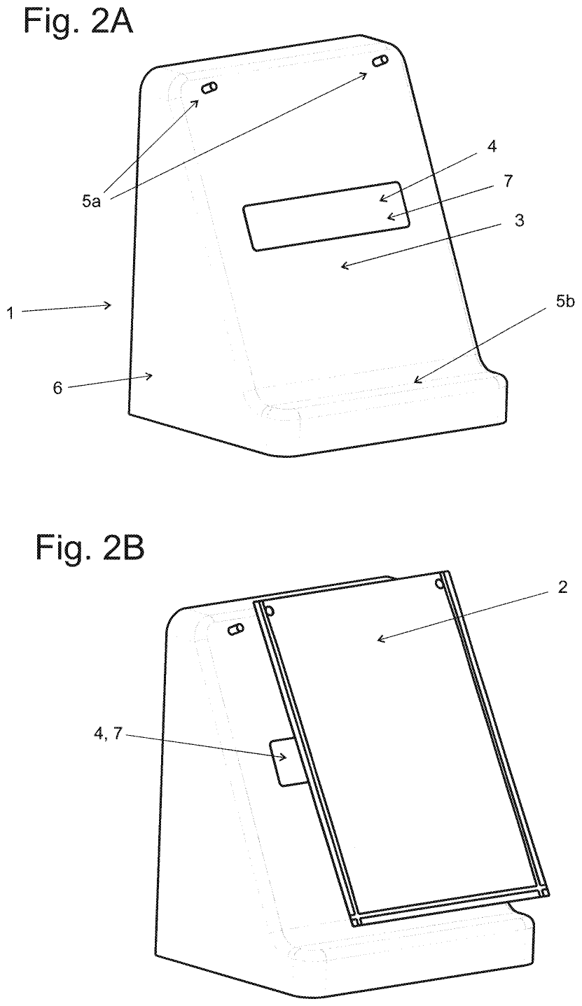

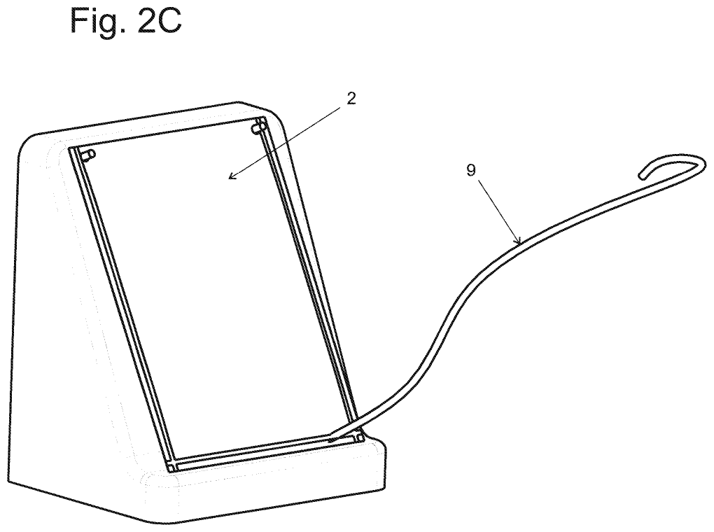

FIG. 2A-C show a second embodiment of a system according to the invention without the bag placed at the receptacle, in the phase of arranging the bag and with the bag finally placed on the receptacle.

The first embodiment shown in FIG. 1A/B is a system 1 for observation and monitoring of media dissolution and/or bacterial growth in a transparent bag which has a receptacle 3 in the form of a substantially flat horizontal upper surface of a housing 6. The bag 2 is shown in FIG. 1B in a position placed on the receptacle 3. The system includes a light emitting device 4 which has a light source (not shown) arranged in the housing and a window 7 through which light can be transmitted from the light source in the housing 6 into and/or through the interior of the bag 2 supported on the surface of the receptacle 3 including the window 7. The light-emitting device accordingly creates a backlit light area at the central portion of the bag 2 and the light transmitted through the bag that can be visually seen on the top side of the bag by the user can be used as an indication of the presence of particles.

The amount of reflected or diffused light can accordingly serve to detect whether a concentrated liquid solution that blocks light or a powder or a granulated material is still present in the interior of the bag and is not dissolved yet. It may also serve as an indication of the presence of bacteria in an otherwise transparent liquid.

Although not shown the size of the window 7 or light emission surface of the light-emitting device 4 can be such that it covers a large, preferably the entire surface area of the bag held at the receptacle. It can even be larger than the size of the bag intended to be placed on the receptacle. Although not shown the light emission surface or window may have the form of plural stripes, dots, rings, triangles, discs, rectangles or others shapes and various combinations and patterns thereof distributed over the surface of the receptacle where the bag is to be placed. If the light emission surface of the light emitting device is larger than the size of the bag actually placed on the receptacle the system may include a function to switch off portions of the light-emitting device to adapt the size of the light emission surface to the size of the bag. The light source of the light-emitting device may be an array of LEDs, an LED panel or a LED module or an OLED panel. The light source may be covered with a diffusing screen which could also include a filter for determining a light spectrum transmitted into the bag. The light source itself may be configured such that it can be adjusted with respect to the wavelength and/or the intensity of the light it emits towards the bag.

A light sensor device can be arranged to receive at least part of the light transmitted into and/or through the interior of the bag supported at the receptacle, preferably a part of the light that is diffused, refracted and/or reflected by the content of the bag. Such light sensor device can be in the form of a single sensor or in the form of an array of plural sensors arranged and distributed over the surface of the bag. The sensors may be placed on the side of the bag that is arranged on the receptacle and that is adjacent to the light emitting device or maybe arranged on the opposite side to the supporting side with the bag placed in between.

In other words, the light sensor device can be arranged on the same side as the light-emitting device or on an opposite side with respect to the major surfaces of the bag or on both sides.

The overall surface of the receptacle in contact with the bag can be used to warm or cool or to regulate the bag temperature to avoid undesired effects on the media in the bag due to the environment of the bag. A suitable device for generating and transmitting heat to and/or from the bag supported at the receptacle by convection, radiation and especially conduction can be any suitable heating device with a heat source including an electrical heater or a heat pipe system including a temperature transport medium circulating in conduits placed adjacent to the receptacle 3 so as to allow the heat transport to and from the bag. The device for generating and transmitting heat to the bag may also be the light emitting device itself if only heating is desired. A filter can be used and selected to modify the wavelength of the light-emitting device in order to promote or avoid the heating effect on the bag or its content. Also, the light intensity and/or frequency of the light source can be modulated to modify the heating power introduced into the interior of the bag.

One or more temperature sensors can be provided as a temperature sensor device to detect the temperature of the bag and/or of the media in the bag supported at the receptacle.

The second embodiment shown in FIG. 2A-C is similar to the first embodiment and differs essentially only in that the receptacle 3 for the bag is formed by a flat but inclined surface of the housing 6. The bag 2 is shown in FIG. 2C in a position placed on the receptacle 3 in a nearly vertical orientation. In order to avoid slipping down of the bag 2 the bag is attached and suspended, at its upper end, at a pair of pins 5a inserted into mating holes on a peripheral edge of the bag. The opposite edge of the bag in the vertical direction is supported on a lower end 5b of the supporting surface. The rectangular window 7 of the light emitting device 4 is arranged substantially in the center of the supporting surface in the vertical and horizontal direction. It may of course have any size and structure as described in connection with the first embodiment.

Another fixation of the bag 2 in the suspended state on the supporting surface like a bracket or a clamp for removable attachment of the bag is possible instead of using the pins 5a engaging the mating holes on the bag 2.

The receptacle 3 may also be formed to hold the bag in a substantially vertical orientation, for example in a gap between two spaced apart holders, of which one is provided with the light emitting device and the other is at least partially transparent to the light if visual inspection by a user from outside is desired.

The system may be provided with a suitable structure 8 to guide one or more tubing 9 connected to the bag placed on the receptacle to allow supply or discharge of fluid to/from the bag while placed on the receptacle of the system.

Although not shown in the drawing the system can be provided with a moving device functionally associated with the receptacle to impart a motion to the bag 2 and/or the media in the bag 2 supported at the receptacle 3. The moving device may be in the form of a motorized mechanical mechanism tilting or moving the receptacle in a predefined motion pattern or deforming the surface of the bag. It may also be in the form of a stirrer previously placed in the bag and moved, preferably by rotation, through magnetic interaction with an external driver in the housing of the system. The moving device can promote dissolution and avoid stagnant zones in the interior of the bag. It may also avoid local temperature differences due to the heat introduced by the light emitting device.

The system of the invention may comprise a control device configured to receive the output of the light sensor device(s), if provided, and/or of the temperature sensor(s), if provided, and to analyse the data, preferably over time, and to output a warning signal and/or to visualize the data and/or to control operation of the components including the light emitting device, the device for transmitting the heat to or from the bag and the moving device. The inclusion of a control device allows an unattended automatic operation of the system from an initial placement of the bag at the receptacle of the system until complete dissolution. It also allows, in another mode, the monitoring of the media in the bag and the automatic detection of bacterial growth. The detection results can be informed to the user through a warning signal to alert the user. The detection results can be visualized in any desired form on a display included in the system or on external devices connected, through data exchange, with the system. The control device may be configured to analyse and monitor the light and/or temperature distribution over at least part of the supporting area of the receptacle. Especially in combination with plural light and/or temperature sensors installed to inspect the bag at different positions the control device may generate an evaluation result that allows the user to locate zones in the bag where dissolution is still incomplete or where bacterial growth takes place. The control device may also be connected to the light emitting device and/or the heating device and/or the moving device in order to automatically switch these devices on/off in accordance with a program implemented in the control device to avoid local overheating or an undesired temperature distribution in the bag or to control the dissolution progress. The program may also include a function to maintain the content of the bag in a certain predefined temperature range.

The receptacle 3 in the first and second embodiments is shown as a flat support surface integrally formed as a part of the housing. It may alternatively include a separate plate or a grid supported on the housing for supporting the bag in a horizontal or an inclined (including vertical) orientation. This structure is preferred where the moving device is provided because the moving device can be arranged to cooperate with such separate plate or grid to impart the mechanical motion to the bag without influence on the light emitting device. Of course, the light emitting device can be included in the separate plate if desired.

1. A system (1) for observation of media dissolution and/or bacterial growth in a transparent bag (2), comprising:
a receptacle (3) for supporting the bag (2);
a light emitting device (4) arranged to transmit light into and/or through the interior of the bag (2) supported at the receptacle (3).

2. The system according to claim 1, wherein the light emitting device (4) is arranged to transmit the light from one or more areas of a supporting side of the receptacle (3) intended to be in contact with the bag (2) to preferably form a backlighted surface, or towards one or more areas of the supporting side from the opposite side with the bag (2) placed in-between.

3. The system according to claim 1, wherein the light emitting device (4) is configured to be adjusted with respect to wavelength and/or intensity of emitted light.

4. The system according to claim 1, wherein the light emitting device (4) comprises a filter for determining the light spectrum transmitted into the bag (2).

5. The system according to claim 1, wherein the light emitting device (4) comprises an area light source or plural light sources distributed over an area, preferably including a LED or OLED light source.

6. The system according to claim 1, comprising a device for transmitting heat to and/or from the bag (2) supported at the receptacle (3).

7. The system according to claim 6, wherein the device for transmitting heat is the light emitting device (4).

8. The system according to claim 1, wherein the receptacle (3) is associated to a moving device to impart a motion to the bag (2) and/or the media in the bag (2) supported at the receptacle (3).

9. The system according to claim 1, comprising a sensor device arranged to receive at least part of the light transmitted into and/or through the interior of the bag (2) supported at the receptacle (3), preferably light that is diffused, refracted and/or reflected by the content of the bag.

10. The system according to claim 9, wherein the sensor device is located on the side of the receptacle (3) in contact with the bag (2) and supporting the bag (2) and/or on an opposite side with the bag (2) placed in-between.

11. The system according to claim 1, comprising a temperature sensor arranged to detect the temperature of the bag (2) and/or of the media in the bag (2) supported at the receptacle (3).

12. The system according to claim 9, comprising a control device configured to receive the output of the sensor device, if provided, and/or of the temperature sensor, if provided, and to analyse the data, referably over time, and to output a warning signal and/or to visualize the data and/or to control operation of the light emitting device and/or of the device for transmitting the heat to the bag (2).

13. The system according to claim 12, wherein the control device is configured to analyse the light and/or temperature distribution over at least part of the supporting area of the receptacle (3).

14. The system according to claim 1, wherein the receptacle (3) includes a plate or a grid for supporting the bag (2).

15. The system according to claim 1, wherein the receptacle is configured to support a bag having a volume of at least 1 liter, preferably at least 10 liter, preferably at least 20 liter.

16. The system according to claim 1, wherein the media in the bag (2), the dissolution of which is to be monitored, is a concentrated liquid solution or a powder or a granulated material.
